(19) United States
(12) Patent Application Publication
(10) Pub. No.: US 2002/0058030 A1
(43) Pub. Date: May 16, 2002

(54) Whole blood separator apparatus and method of use

(75) Inventors: Rodney L. Monroy, Rockport, MA (US); David N. Cook, Lexington, MA (US); Walter C. Ogier, Winchester, MA (US); Robert J. Schmittling, Cambridge, MA (US)

Correspondence Address: Leo R. Reynolds, Esq., HAMILTON, BROOK, SMITH & REYNOLDS, P.C., Two Militia Drive, Lexington, MA, 02421-4799, US

(73) Assignee: Eligix, Inc., Medford, MA (US)

(21) Appl. No.: 09/848,545

(22) Filed: May 3, 2001

Related U.S. Application Data
(60) Provisional application No. 60/201,515, filed on May 3, 2000.

(51) Int. Cl. A61K 39/395; A61M 37/00; B01D 61/00

(52) U.S. Cl. 424/140.1; 435/2; 604/6.02; 210/645

(57) ABSTRACT

An apparatus for separating a selected cell population from blood or a blood component includes a container, a plurality of particles having a reactant that binds to a selected cell population and a separating device for separating the selected cell population from the blood or blood component. The particles have a density that allows gravity settling of the particles through the blood or blood component. The separating device can include a device for compressing the container, a magnet or a filter, for example. The apparatus can also include a mixing device for mixing the particles with the blood or blood component. The reactant bound to the particles can include an antibody or antibody fragment, such as lectin or a lectin fragment.

### RELATED APPLICATION

This application claims the benefit of U.S. Provisional Application No. 60/201,515 filed on May 3, 2000. The entire teachings of this application is incorporated herein by reference.

### BACKGROUND OF THE INVENTION

When blood is collected from a donor for use, the whole blood is typically separated into several components including erythrocytes, or red blood cells, thrombocytes, or platelets, and plasma. Commonly, individual blood components are used therapeutically, rather than administering whole blood, in order to maximize the clinical and economic utility of blood. Whole blood includes leukocytes or white blood cells that are carried, during processing, into each of the blood components. The white blood cells in many instances are ultimately filtered out of the blood components to reduce patient exposure to this cell type. Removal is desirable since white blood cells may transmit infectious agents, such as cell-associated viruses (e.g. cytomegalovirus or human immunodeficiency virus) or they may cause adverse immunological reactions, such as alloimmunization. In recent years, a theoretical concern over blood-borne transmission of prion diseases (e.g. Creutzfeld Jacob's Disease) has provided additional motivation to remove leukocytes from blood products.

Blood is collected using both manual and automated devices. One conventional manual apparatus for collecting and processing whole blood utilizes a system of flexible containers connected by tubing. Whole blood from a donor is collected in a primary container that includes an anti-coagulant solution to prevent clotting of blood. Typically, approximately 500 milliliters of whole blood is collected. The primary collection container and associated containers are spun in a centrifuge to separate red blood cells, platelets and plasma, which differ in density or size. The use of centrifugal methods inevitably results in some level of white blood cell contamination in each of the blood components. The white blood cells may be physically trapped, for instance among the far more numerous red blood cells, or they may be distributed according to their density, for instance, with the platelets. In addition, incomplete centrifugation or formation of white blood cell fragments can lead to the presence of white blood cells in the plasma.

If the whole blood is centrifuged at low speed (e.g. 300× g) the blood separates generally into a red blood cell portion and a portion containing both plasma and platelets (so-called platelet-rich plasma). The platelet-rich plasma is transferred using a device called an expressor into a first associated container through interconnected tubing by compressing the primary collection container to express the upper layer of plasma and platelets into this first associated container. A red blood cell preservative solution, located in a second associated container may then be transferred into the primary collection container with red blood cells, creating a so-called packed red blood cell solution. The container of packed red blood cells is physically separated from the associated containers by creating a heat seal in the connecting tubing. The first associated container is spun again at higher speed to pellet the platelets (and most of the attendant white blood cells) and separate them from the plasma. The plasma can then be expressed into an empty second associated container to produce the separate blood components, packed red blood cells, platelet concentrate and fresh frozen plasma.

If the whole blood is centrifuged at high speed (e.g. 1300× g) the whole blood separates generally into three portions, a red blood cell portion, a buffy coat portion containing platelets, red blood cells and a large portion of the white cells, and a plasma portion. The primary collection container is placed into an expresser. The expresser typically has two opposing plates, one of which may be notched to accommodate the buffy coat portion. The expresser may be automated or manual. The second plate compresses the upper and lower portions of the container, the plasma portion and red blood cells portion, respectively; each portion is moved to a respective associated container through interconnected tubing by compressing the collection bag. The associated container for red blood cells will usually contain a red blood cell preservative solution. Upon separation of the individual containers, the buffy coat portion is further processed by centrifugation to effect some separation of the platelets from the contaminating white and red blood cells. This high speed centrifugation method, like the low speed method, ultimately results in three separate containers each containing one of the three parts: red blood cells, plasma and platelets.

After fractionation using the apparatus described above, the individual blood components are often further processed to reduce the level of white blood cells or leukocytes that are distributed in each of the red blood cells, platelets, and the plasma. White blood cell removal is effected with a mechanical depth filter through which each portion is passed and typical takes 20 or more minutes to filter the red blood cell component. Specialized filters are used to achieve the maximum yield of each blood component.

Automated apheresis devices also exist which manufacture blood components in real time during the blood donation. The donor is connected via a needle and sterile tubing to the apheresis device. Donor blood is mixed with a controlled ratio of anticoagulant and is sequestered into a sterile disposable container system. The container system creates a closed loop between the donor and device such that one or more of the desired blood component(s) is separated by centrifugation, and the remaining blood is returned to the donor. An advantage of this method is that a higher yield of the desired blood component(s) can be obtained while avoiding donor hypovolemia. Some automated devices enable in-process leukodepletion, such that post-apheresis reduction of white blood cells is not required. In other instances (e.g. the automated collection of red blood cells), the current art does not sufficiently separate the therapeutic blood component from the undesirable white blood cells. In these cases, further processing to reduce the number of white blood cells is desirable.

### SUMMARY OF THE INVENTION

An embodiment of the invention relates to an apparatus for separating a selected cell population from blood or a blood component. The apparatus includes a container for receiving the blood or blood component, a plurality of particles having bound thereto a reactant which specifically binds to the selected cell population and a separating device for separating the selected cell population from the blood or blood component. The particles have a density sufficient to provide differential gravity settling of the population or subpopulation from the sample where the particle density is at least two times the density of the cells.

The separating device can include a device for compressing the container, a magnetic device for retaining the particles, a sizing filter for retaining the particles, a flow path allowing the particles to settle from the blood or blood component or a centrifugal device for retaining the particles after differential settling. The separating device can also include a centrifugal device for separating the particles from the blood or blood component during differential settling.

The apparatus can include a device for mixing the particles and the blood and a means for dispersing the particles in blood. The apparatus can also include a means for retaining the particles prior to introduction into the blood or blood component. The blood component held by the container can be whole blood, red blood cells, platelets or plasma.

The container can include a fluid transfer device for removing fluid from the container. The container can generally be rigid or can be flexible. For the flexible container, the separating device can include a device for compressing the container. The container can include a fluid transfer device for removing a fluid from the container. The container can be a sterile container wherein the particles are sterilized within the container. The apparatus can also include a secondary storage container in communication with the container.

The particles can be formed of a nickel material. The particles can have a diameter from about 3 to 35 microns, preferably about 5 microns. The particles can have a density of about 7-10 g/cm³. The particles can also have a density of about three times the density of the cells of the blood. The reactant or ligand that coats the particles can be an antibody or a fragment thereof. The antibody or antibody fragment can specifically bind to white blood cells. The antibody or antibody fragment can also be an anti-CD45 antibody or can be a pan-leukocyte antibody. The reactant or ligand can also be a lectin or a fragment thereof.

Another embodiment of the invention relates to an apparatus for separating a selected cell population from blood or a blood component. The apparatus includes a flexible primary collection container for receiving the blood or blood component, a plurality of particles in the primary collection container, the particles having bound thereto a ligand which specifically binds to the selected cell population, at least one secondary flexible container, a plurality of fluid transfer devices interconnecting the flexible containers and a compression separation device for separating the selected cell population bound to the particles from the blood or blood component. The fluid transfer devices move at least a portion of the blood through at least one of the tubes to at least one of the associated flexible containers.

The primary collection containers and secondary collection containers can be sterile. The separating device further can include a secondary means for retaining the particles after settling, such as a magnetic device, a sizing filter, a flow path or a centrifugal device.

The apparatus can also include a device for mixing the particles and the blood or a centrifuge for spinning at least the primary flexible container at a speed to assist in the settling of the particles from the remaining blood or blood component.

The primary flexible collection container can include a connection to a blood source where the blood source is a donor, a blood collection apparatus or an infusion apparatus, for example.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic block diagram of a whole blood separator method according to the present invention;

FIG. 2 is a schematic diagram of components of an apparatus according to the present invention;

FIGS. 3A and 3B are front and side views of a blood container mixer of the present invention;

FIG. 4 is a secondary serpentine separator;

FIG. 5 is a conceptual embodiment of a particle with targeted cells bound thereto in accordance with the present invention;

FIG. 6 shows an alternative flexible container with a chamber for retaining the particles prior to dispersion within the blood in the flexible container;

FIGS. 7A-7F schematically show a process of separating whole blood into four components;

FIGS. 8A-8E schematically show an alternative process of separating whole blood into components;

FIG. 9 is a perspective view of a rigid collection container;

FIG. 10 is a chart comparing pre and post depletion samples from experiments of depletion of neutrophils from whole blood; and

FIG. 11 is a table comparing pre and post depletion samples from flexible and rigid containers.

### DETAILED DESCRIPTION OF THE INVENTION

The state of the art for leukoreduction utilizes depth filters constructed from fibrous material encased in a plastic housing. White blood cells are retained by the filters through a variety of mechanisms. One mechanism comprises simple mechanical filtration or trapping of the leukocytes in the filter. Therapeutic blood components are less likely to be trapped due to their greater flexibility (red blood cells) or smaller size (platelets and plasma proteins). Another mechanism relies upon the unique biological properties of white blood cells, which causes them to preferentially adhere to a fiber with the requisite chemical and physical properties.

Current filters have certain significant limitations. First of all, they lack the desired selectivity for white blood cells over therapeutic blood components. An ideal leukoreduction device would be used prior to fractionating whole blood, eliminating the need for specialized filters and individual procedures for each blood component. Currently, whole blood filters deplete both white blood cells and platelets, thus removing one of the therapeutically and economically important fractions of blood. For this reason, specialized blood filters are used at the end of the fractionation procedure to remove white blood cells. Thus, there exists a need for a device capable of removing white blood cells selectively while obtaining high yields of red blood cells, platelets and plasma.

Another disadvantage of current filters is their tendency to clog during filtration. Blood is a corpuscular medium and, in addition to cells and clumps of cells, it can contain globules of fat. Furthermore, if blood is insufficiently anti-coagulated during collection, microscopic and even macroscopic clots can develop prior to further processing. Insufficient anticoagulation is a common problem during donation. As a result, depth filters can clog during leukofiltration. If a filter clogs, the processing technician has the choice of discarding the affected blood unit, or replacing the failing filter with a new one. This increases the costs of both labor and materials in producing a blood component. Thus, there exists a need for a leukoreduction device that avoids the problem of filter clogging.

Filters remove white blood cells, in part, because the white blood cell conforms to the surface of the fiber filter. However, immediately after collection of whole blood, white blood cells begin to degrade and fragment into vesicles, a process that continues over the subsequent days and weeks of storage of the blood component. Blood filters do not effectively remove these fragments because the vesicle cannot readily conform to the fiber filter. These vesicles may still possess cellular antigens and thus be capable of alloimmunizing the patient. Thus, there exists a need for a device that can remove antigenic white blood cell fragments as well as intact white blood cells.

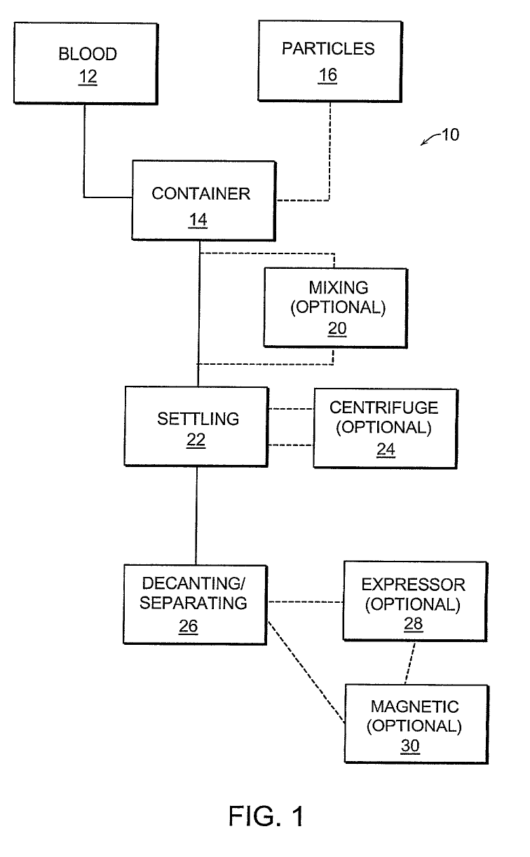

A description of preferred embodiments of the invention follows. Referring now to FIG. 1, a block diagram of the method of use of the leukoreduction apparatus is shown. The leukoreduction apparatus 10 includes a source of blood 12, typically a blood donor or a container containing a unit of whole blood or a blood component. The blood 12 is transferred via tubing to a container 14 such as a primary collection container. The sterile container holds particles 16, or is connected to a satellite container holding particles 16, which is further described below. In one preferred method, the particles 16 are added to the container 14, either before or after the transfer of the blood into the container 14.

The plurality of particles 16 and the blood 12 are combined within the container 14 such that the particles are dispersed generally uniformly through the blood 12 within the container 14, as described below.

The particles 16 include a ligand capable of binding specifically to selected cells, such as leukocytes, in the blood or blood component. Possible ligands include, for example, lectin proteins or monoclonal or polyclonal antibodies such as pan-leukocite antibodies, anti-CD45 antibodies or any other molecule capable of binding with the requisite affinity and specificity to the selected cells. Functional fragments of any of these ligands can also be utilized. The ligand can be bound to the particles 16 directly, either covalently or by adsorption, or indirectly via an antibody in any conventional manner. The particles 16 have a density sufficiently greater than the density of the cellular populations in the blood 12, both targeted (i.e., white blood cells) and non-targeted (i.e. the red blood cells and platelets) such that the particles 16 and the cells bound thereto settle differentially through the blood 12. Preferably the particles settle solely under gravity and this action is sufficient to separate them from the non-target cells. For example, if the blood cells have a density on the order of 1.05 gm/cc, then the particles 16 can be substantially more dense than the cells, at least on the order to two (2) to three (3) times more dense than the cells. The particles 16 are further described in U.S. Patent Application No. 08/556,667 filed Nov. 13, 1995 and U.S. Pat. No. 5,576,185 which issued on Nov. 19, 1996, the entire teachings of which are incorporated herein by reference.

The particles 16 preferably are made with a nominal diameter of about five (5) microns with a preferable range of three (3) to thirty-five (35) microns, but not limited thereto. The fines (smaller fragments) are eliminated prior to utilization. The density of the particles 16 can be at least approximately two (2) to three (3) times the density of the cells to be selected from a cell population. The preferred particles are relatively heavy, having a density typically on the order of seven (7) to ten (10) gm/cc. Dense materials such as metals, glass or high density plastics can be used to form particles. The density of the particles is selected such that the particles differentially settle through the sample suspension more rapidly than the cells. Although no specific type of particle 16 is critical, a paramagnetic high density particle 16 is preferable. In some cases, however, ferromagnetic particles, such as nickel can be preferable. One preferable particle 16 is formed from nickel, such as nickel powders made by INCO as Nickel Powder Type 123. Paramagnetic high density particles can also be formed from iron.

In one preferred embodiment, the combined sample portion and the particles 16 are mixed by rotating the container 14 as shown by block 20, and further described below. The blood 12 and the particles 16 are mixed to facilitate the rapid binding of the particles 16 to the selected blood component of interest, which in a preferred embodiment is the leukocytes or white blood cells. The mixing of the sample 12 and the particles 16 is effected to cause the particles 16 to rapidly and frequently contact the selected cells in the sample 12. During the preferred mode of mixing, the particles 16 repeatedly pass or settle through the sample to bind to the target cells without substantially physically damaging the cells.

To those skilled in the art of blood component manufacturing, storage and transfusion, substantial cell damage is understood to mean that the blood component has lost the therapeutic properties for which it was intended. Accepted medical practice defines a large range of cellular, biochemical and physical properties of blood cells that are therapeutically acceptable. For instance, regulatory standards of product approval recognize that blood cells with far less than 100% activity can be therapeutically useful. For instance, 24-hour post transfusion recovery of red blood cells as low as 70% after processing and storage has been deemed an acceptable value for therapeutic use. Standard in vitro methods for assessing red blood cell function are well-known to those skilled in the art, including measures of hemolysis, ATP levels, cellular deformability and pH. Similarly, platelet recoveries 24-hour post transfusion of 30-50% are considered routine and acceptable in transfusion medicine. Standard in vitro methods for assessing platelet function are well-known to those skilled in the art, including coagulation assays, pH, shape change, osmotic shock and morphology.

In a different embodiment, the initial dispersion of the particles in the blood 12 leads to sufficient contact between the particles and the selected cells, and no additional mixing step is required. An advantage to using the dense particles 16 is that they differentially settle through the sample 12 under the influence of gravity, leading to multiple cellular contacts, without substantial trapping of non-selected or non-targeted cells. In this embodiment, the high rate of movement of the particles 16 settling through the blood 12 obviates the necessity for mixing.

After the particles 16 are dispersed through the blood 12 or have been mixed with the blood 12, the particles 16 are allowed to settle to the bottom of a container 14 as illustrated by the block 22. The gravity settling effectively separates the target cells from the non-target cells.

In one preferred embodiment, the blood or blood component 12 is expressed away from the particles 16 bound to target cells using elements of the separation apparatus 10. Decanting 26 is done using an expresser 28 comprising one of several different configurations as described in further detail below. One such apparatus is a separating device or expressor, represented by block 28, in which the container 14 is compressed, therein reducing its volume and forcing the blood, with the white cells removed, out of the container 14. Typically the blood 12 is expressed through a fluid transfer device, such as a tube, to another container as further described below. The particles 16 with bound target cells are generally retained at the bottom of the container 14 due to their greater density.

The device as described herein can optionally have a secondary means for insuring the separation of the desired non-target blood cells from the particles 16 with bound target cells. The purpose of the secondary capture step is to further reduce the probability that particles 16 can pass into the final processed blood component or components. The nature of this secondary capture takes advantage of specific properties of the particles 16. For instance, if the particles are made of or incorporate a magnetized, ferromagnetic, or paramagnetic substance, they can be retained, in combination with the expressor 28, or separately, by use of a magnet. This preferred embodiment of the apparatus method is represented by block 30. Depending on the type of container 14, the expressor, representated by block 28 can be used in combination with the magnet, or if the container 14 is more rigid, the container can be rotated with the magnet held at the bottom, to allow the remaining non-target blood 12 to pour or drain from the container 14.

Another optional secondary means of retaining the particles during the decanting process can rely on the size or rigidity of the particles relative to the blood component. If the more rigid particles are large relative to the red blood cell, for example 10 microns in diameter or greater, they can be retained by a sizing filter placed at the outlet of the container 14. In particular since red blood cells are known to be highly flexible, and since platelets are sub-cellular fragments, the size differential needed to achieve the separation of the more rigid particles is not necessarily large, i.e. the particles and the red blood cells can, in fact, be of comparable sizes.

Another optional secondary means of retaining the particles is to centrifuge the settled particles. Referring to FIG. 1, while the settling step 22 preferably can be accomplished by gravity separation alone, a centrifugation step can further compact the particles 16 with bound white blood cells in the bottom of container 14. In addition, the centrifugation of the blood 12 and the particles 16 can be at such a rate to separate the blood into layers for fractionation, as described in the conventional blood centrifugation methods above. However, in contrast to those conventional methods, the white blood cells bound to particles are effectively sequestered at the bottom of the container. Preferably, centrifugation is performed after settling. In another embodiment, centrifugation can be performed simultaneously with settling. In this embodiment, centrifugation enhances the rate at which the particles 16 settle and also leads to enhanced compaction of the particles 16 with bound white blood cells at the bottom of the container. Because the particles 16 are at least two (2) times the density of the cells, any centrifugation speed sufficient to separate the cells also enhances the rate of particle settling.

Other optional secondary means of retaining the particles 16 can be envisioned. The flow of the blood over any path that minimizes shear and allows sufficient time for settling can be used. A tortuous path can be combined with the function of a blood warmer to create a dual-function device. A magnetic field can also be introduced into this flow path to further facilitate secondary particle capture.

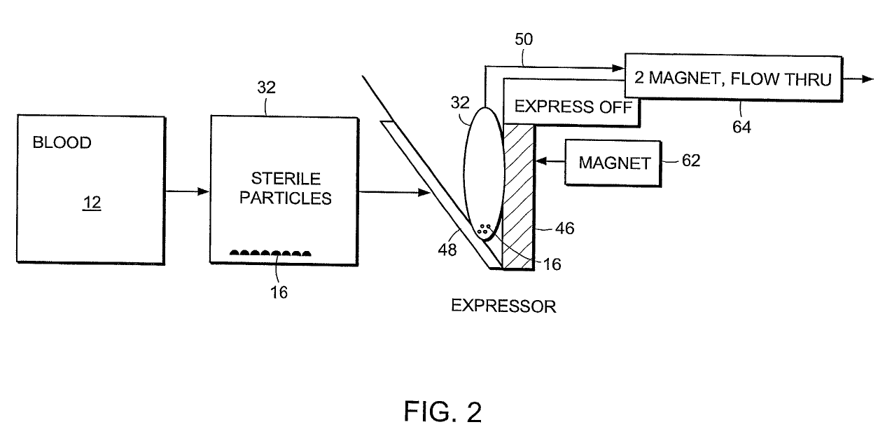

Referring to FIG. 2, a sterile blood container 32 has a plurality of particles 16 which include ligands such as monoclonal or polyclonal antibodies bound thereto. Blood 12 is introduced into the container 32 from another blood container or a donor via a sterile tube.

In a preferred embodiment, the sterile blood container 32 is formed from flexible plastic sheeting that is biocompatible with the blood or blood components 12, such as polyvinyl chloride or polyethylene or other materials known to those skilled in the art of making blood storage containers. The blood container 32, the particles 16 and other components of the separation apparatus 10 that contact the blood 12 directly can be sterilized by controlled heat, ethylene oxide gas or by radiation. The preferred method of sterilization can be selected by one skilled in the art to preserve the activity of the particles 16, particularly the ligand bound thereto, and can be dependent on the physical characteristics, composition and number of particles 16. Preferred sterilization methods also depend on whether the device is “dry,” that is lacking a solution component, or “wet.” Alternatively, it is well known to those skilled in the art of making blood storage containers that individual incompatible components can be separately sterilized by different means and then joined via a sterile connection process that connects two devices via sterile tubing leads. The preferred method of sterilization is a terminal sterilization at the 10-6 Sterility Assurance Level in order to enable extended storage of the blood component after processing in the separation apparatus 10.

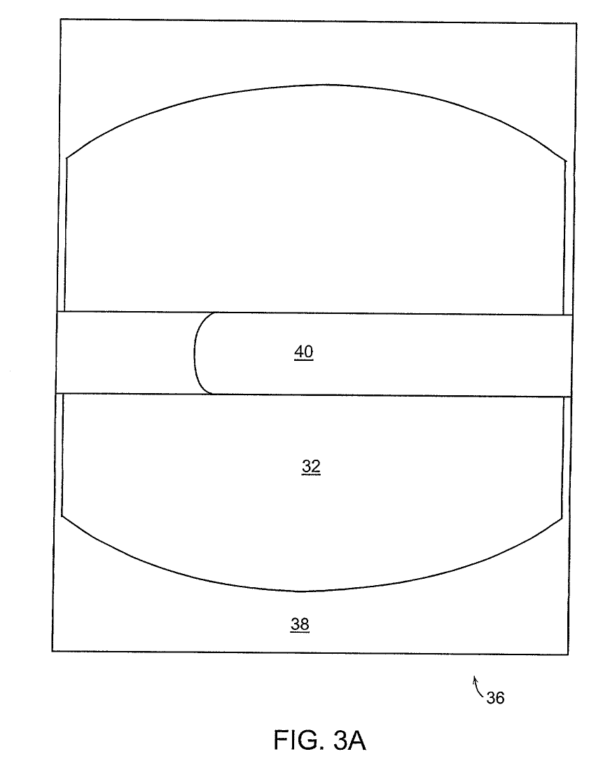

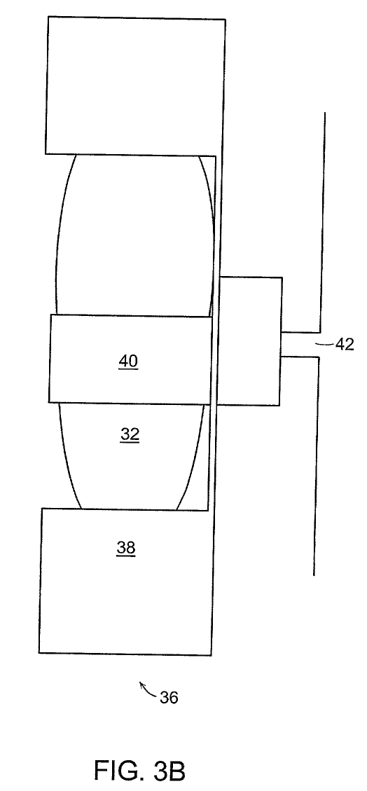

In one preferred embodiment, the blood container 32 is mixed using a mixing device 36, such as illustrated in FIG. 3A and 3B. The mixing device or mixer 36 has a means for fixing the blood container to the mixer 36, such as a pair of pockets or receptacles 38 for retaining the upper and lower portions of the blood container 32. An alternate means for fixing the blood container 32 to the mixer 36 can include utilizing cutouts on the manufactured flaps or tabs commonly found on blood containers 32. The cutouts can affix the container 32 to the mixer 36. A holding device 40, such as a flexible strap, can secure the blood container 32 within the fixing device 36. During operation, the mixing device 36 is rotated about a shaft 42. The mixer 36 is mounted substantially vertically to provide a desirable end over end tumbling of the blood 12 and the particles 16. It is recognized that the mixer 36 can be mounted horizontally and that other style mixing devices that achieve the rotation of the blood 12 and particles 16 at a substantial speed to allow proper mixing without damaging of the components can be used. In a preferred embodiment, the blood container is rotated at a rate of approximately 16 revolutions per minute and the particles 16 are caused to settle through a substantial portion of the sample on each rotation to bond to the target cells (i.e. the white blood cells) in the blood 12.

One preferred method of mixing the particles 16 with the blood 12 is to gently tumble the particles 16 and sample mixture end over end causing the particles 16 repeatedly to fall through the blood 12 to bind to the target cell population of interest. This can be accomplished using a rotisserie-like movement of the container 32. Alternately, roller rocking or stronger mixing procedures can also be effective, if physical damage to the cells of interest by the dense particles 16 is avoided. One such device can be a test tube holder that rotates slowly to rotate the test tube or similar vessel end over end. This allows a “gentle mixing” of the particles 16 and blood 12 in which the particles 16 mix and subsequently settle through a substantial portion of the sample on each rotation, allowing the targeted cells to bind to the particles with no apparent physical damage to the cells. The same mixing motion can be obtained by rotating or oscillating the tube back and forth with each end being first on top and then on the bottom, similar to the end over end rotation. The speed of the roller rocker also can be set to effect substantially the same mixing procedure.

Referring back to FIG. 2, after the particles 16 have been mixed or dispersed within the blood container 32, the blood container 32 is positioned to allow the particles 16 with the attached white blood cells to settle. The blood container 32 can be left in the mixer 36 in a stationary vertical position or in the separating device 26 described below, or in other locations to allow settling. In a preferred embodiment, the container 32 is placed in the separating device 26.

The blood container 32 is placed in a separating device 26 such as an expressor 44 shown in FIG. 2. The expressor 44 has a pair of plates 46 and 48 in which one of the plates 48 is moved towards the other plate 46 therein compressing the blood container 32. The expresser may be automated or manual. The blood 12 is expressed through a tube 50 near the top of the container with the settled white cells bounded to particles 16 at the bottom of the container, thereby transferring the blood 12, substantially free of the white blood cells, from the collection container 32 to another container. In a preferred embodiment, the expressor 40 has a magnet 62 located on one of the plates 46 to retain the particles 16 within the blood container 32. The particles 16 in this embodiment have magnetic properties such that the magnet 62 attracts the particles 16, such that the particles remain within the container 32 during expression.

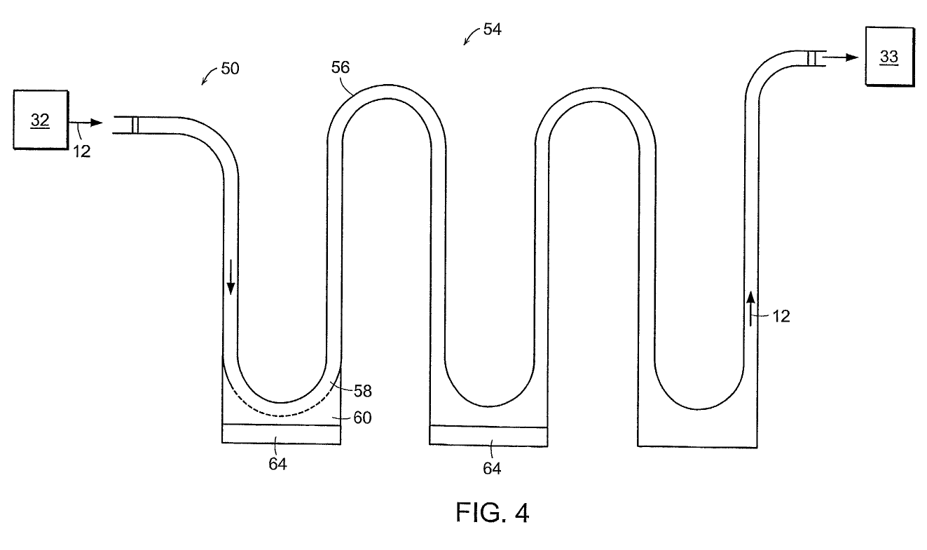

In a preferred embodiment, the tubing 50 (tube) can traverse a flow path, preferably a serpentine path, similar to that used in a blood warming container. Referring to FIG. 4, the separator apparatus 10 includes a serpentine separator 54. The blood 12 flows through the serpentine tubing 56 in generally a vertical path and those particles 16 which inadvertently have left the blood container 32 are separated from the bulk fluid by gravity at the bottom of each curve 58.

In the embodiment shown, the tubing 56 has an optional enlarged trap area 60. Magnets 64 can be mounted adjacent the tubing 50. The magnets 64 are mounted adjacent to the traps 60. Preferably, two magnets are used in the separator 54, however any number of magnets can be used. The blood that flows through the tubing 50 from the blood container 32 during the separation can pass by the magnets 64 to prevent the further flow of particles 16 downstream. The particles 16 are attracted to the magnets 64 and collect within the trap areas 60. The blood can collect in at least a secondary collection chamber or storage container 33.

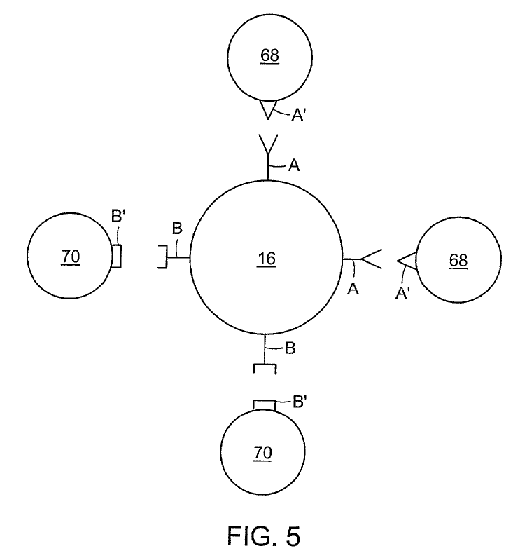

Referring now to FIG. 5, a conceptual diagram illustrates one particle 16 having two different antibodies A and B bound thereto. For example purposes, a pair of A positive cells 68 are illustrated, including at least one antigen A′, which specifically binds with one bound antibody A on the particle 16. A pair of B positive cells 70 also are illustrated including at least one antigen B′, which specifically binds with one bound antibody B on the particle 16. Generally, there is no particular order to the cell binding and there generally is an A or a B positive cell blocking the view of the particle 16 on both free sides of the particle 16. Also, the A & B antibodies on one particle 16 bind to a single cell expressing both the A′ and B′ antigens. For example, if the A cell was a positive cell and the B cell was a CD8 positive cell, then there would be four or five A cells and only one or two B cells bound to the particle 16 due to the natural ratios of these cell types in whole blood. Although two different antibodies A and B are described as both bound to the particle 16, each antibody can be bound to separate particles 16 as desired. While the above discussion focuses on one particle with different ligands, two particles each with a different ligand can also be used to achieve the same purpose. Examples of possible ligands that could be used for white blood cell removal include anti-CD45 antibody, anti-CD56 antibody and soybean agglutinin, among others. Other antibodies can be used, as described in U.S. Pat. No. 5,576,185, issued on Nov. 19, 1996, (see for example Table 1), the entire teachings of which are incorporated by reference herein.

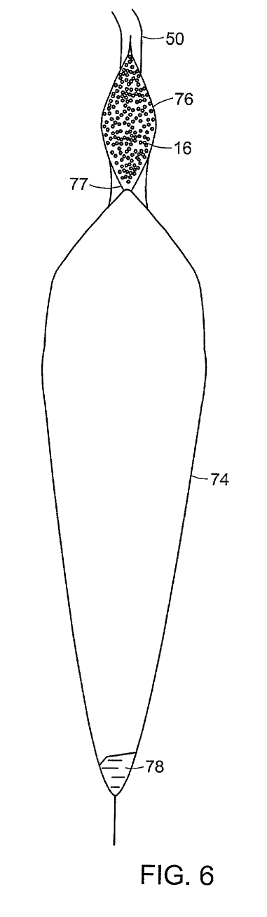

The separator apparatus 10 can include a flexible blood container 74, in a preferred embodiment, which has a separate compartment or chamber 76 that contains particles 16, as illustrated schematically in FIG. 6. A seal 77 between compartments 76 and blood container 74 can be a temporary seal such that the seal 77 prevents particles or liquids from transferring between the compartment 76 and the container 74, but it can be disrupted with an appropriate shearing force. The manufacture of temporary or pealable seals is well known to those skilled in the art of making blood storage containers. The flexible blood container 74 includes a tubing 50 similar to other embodiments for allowing the transfer of blood into and out of the blood container 74. In addition, the blood container 74 can contain an anti-coagulant solution 78.

After the blood 12 is placed within the flexible blood container 74, the compartment 76 is opened by breaking the temporary seal, thereby allowing the particles 16 to disperse or gravity-settle through the blood 12 contained in the flexible container 74. It is recognized that this dispersion of the particles 16 through the blood 12 can obviate the need for the mixing step 20 of FIG. 1. In a preferred embodiment, the particles 16 are allowed to settle through the blood 12 within the flexible container 74 during at least one end-over-end rotation of the container, to insure that the target components (i.e. the white blood cells) are bound to the particles 16.

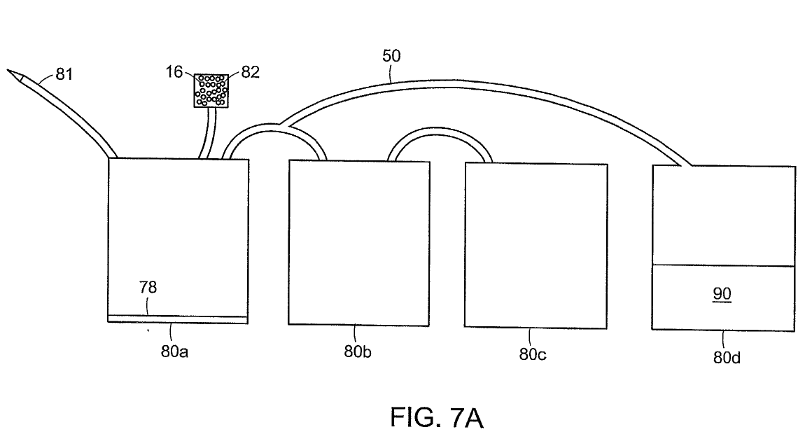

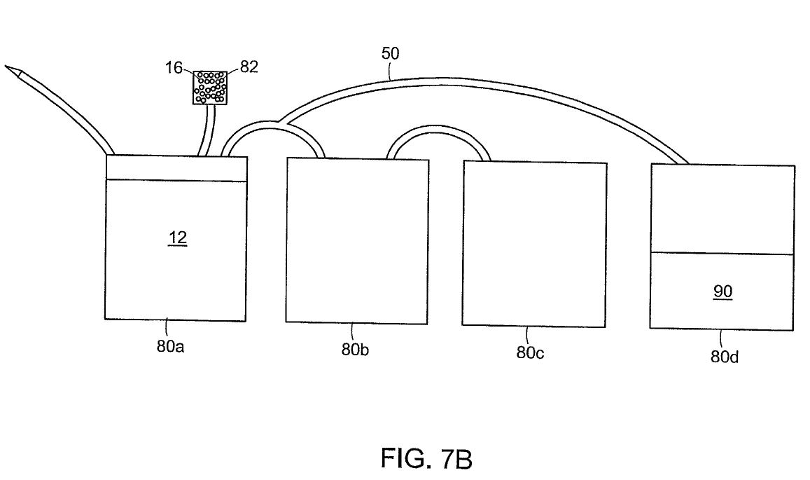

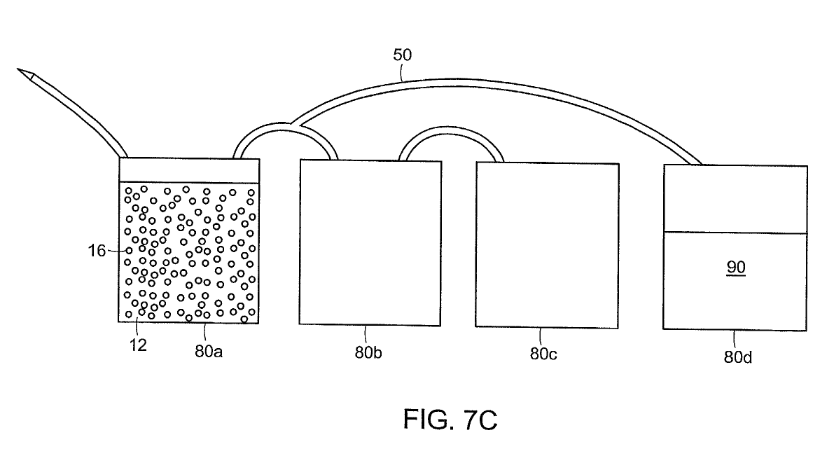

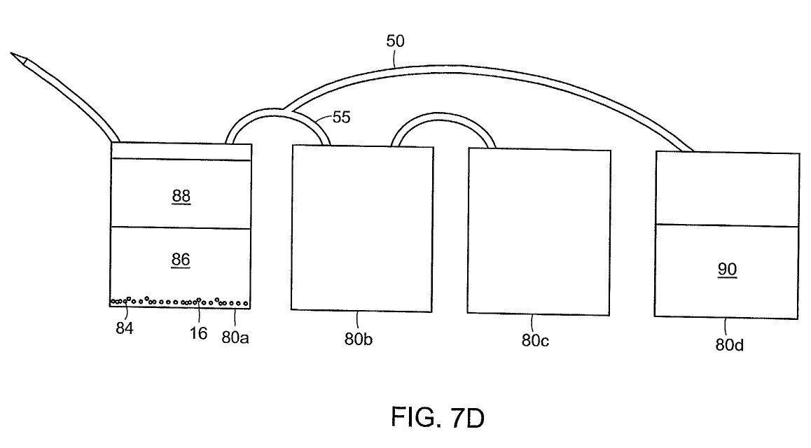

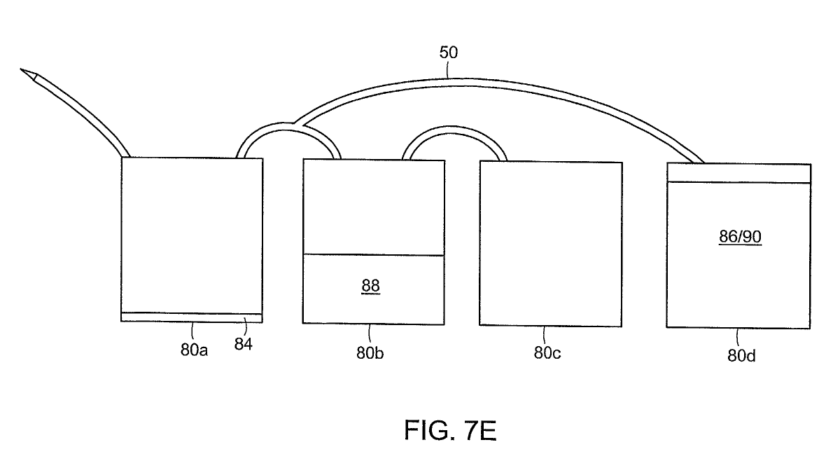

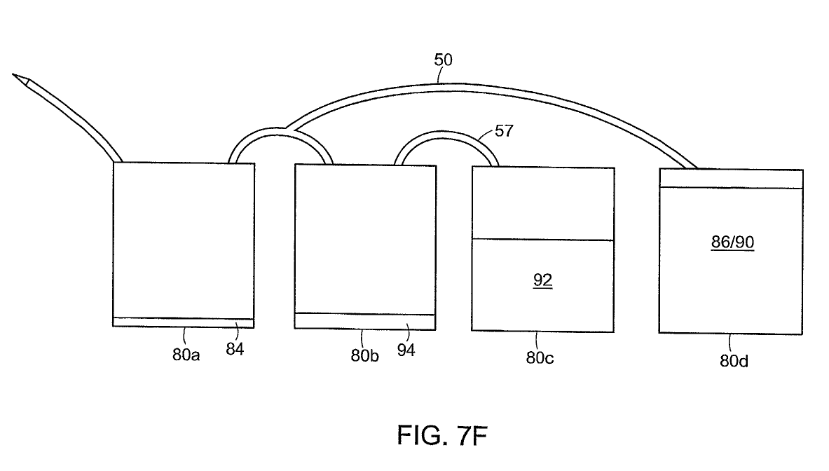

FIGS. 7A-7F show schematically the separation of the blood 12 into four components. Referring to FIG. 7A, a blood collection quad pack 80, according to the invention, is shown. The quad pack 80 has four flexible sterile containers 80a, 80b, 80c and 80d, each of approximately 500 ml. The containers 80 are interconnected by tubing 50 which can be clamped or sealed to the containers 50 as further described below. The first container or primary collection container 80a has tubing and a needle set 81 to allow blood to be drawn from a donor for example and has, within the sterile container, anti-coagulant solution 78. Alternatively, the first container 80a can be sterilely connected to a unit of whole blood or a blood component prepared by apheresis, in which case anticoagulant is not necessary. The first container 80a can also be attached to other blood sources, such as a blood collection apparatus or an infusion apparatus. A storage container or satellite pouch 82 containing particles 16 is connected to the collection container via a tube. The tube 50 can contain a break-away cannula to allow controlled dosing of the particles with the blood.

After the blood 12 from the donor is collected in the collection bag 80a as illustrated in FIG. 7B, the particles 16 are added to the collection bag 80a. At this time, the container is mixed as illustrated in FIG. 7C, using a blood container mixer similar to that disclosed in FIGS. 3A and 3B above, and the particles 16 are allowed to settle. The collection container 80a and the associated other containers 80b-80d, interconnected by clamped sterile tubing 50, are then placed in a centrifuge and rotated at 300-400 times the force of gravity for a time period of approximately 5-15 minutes.

Referring to FIG. 7D, the blood is illustrated as being separated with the particles 16 having attached target white blood cells 84 at the bottom of the container, a layer of red blood cells 86 in the center and a layer of platelets and plasma 88 at the top. After centrifugation of the blood in the device, the collection container 80a is placed in an extractor device, such as the expresser shown in FIG. 2, and compressed such that the plasma and platelets are transferred via a tubing 55 to a first associated container 80b. After the plasma and platelets 88 are moved to the first associated container 80b, the tubing 55 to that container is clamped off and the primary collection container 80a is further compressed to transfer the red blood cells 86 to another associated container 80d which has within it a red blood cell preservative solution 90, as shown in FIG. 7E.

The platelets and plasma are further fractionated as shown in FIG. 7F, according to standard methods. The plasma 92 is transferred into container 80c, by tubing 57 and the platelets 94 are retained in container 80b. The white blood cells 84 and particles 16 which are heavier than the blood are retained in the primary container 80a, thus effectively leukodepleting the blood.

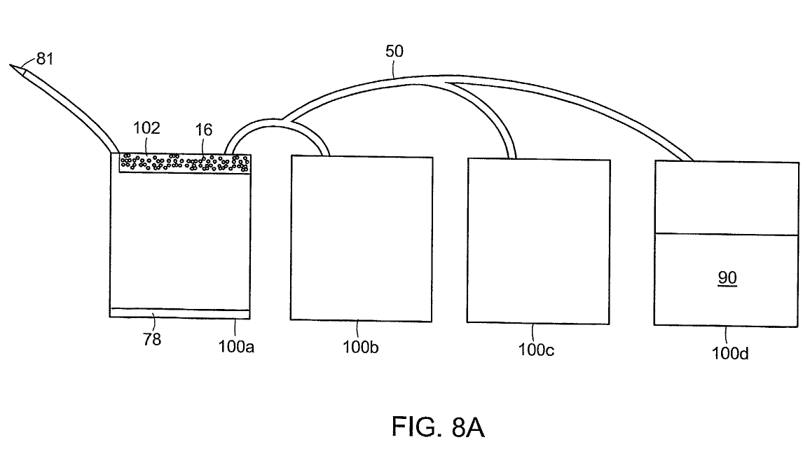

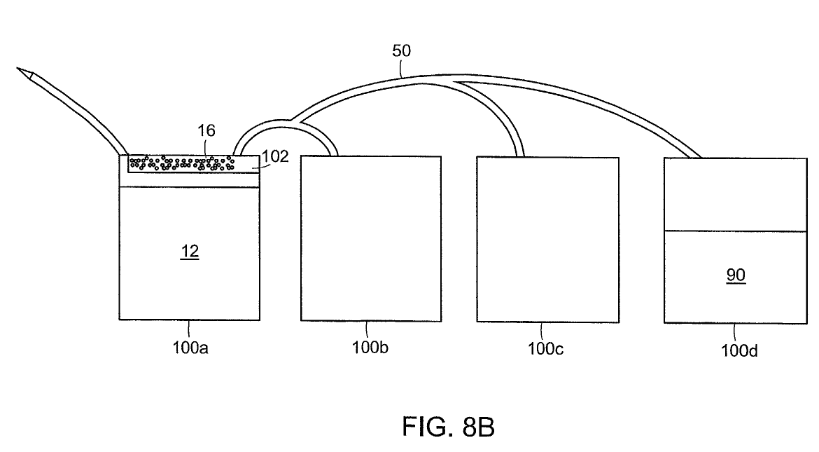

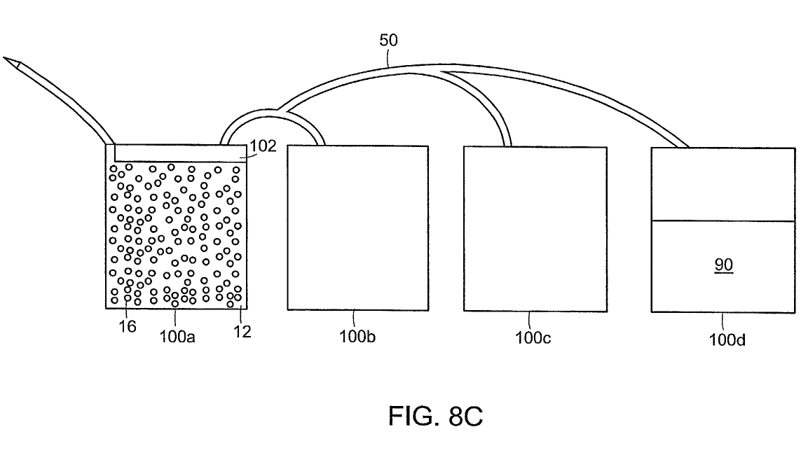

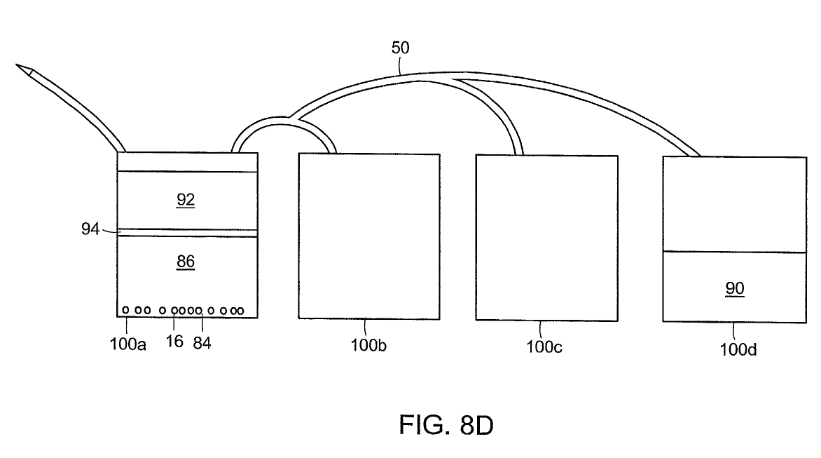

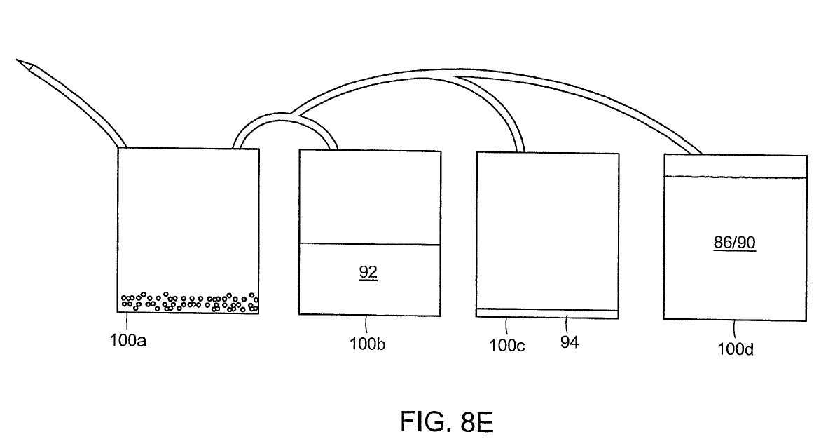

FIGS. 8A-8E show schematically an alternative method of separation of blood 12 into four components. A blood collection quad pack 100 has four flexible sterile containers 100a, 100b, 100c and 100d, each of approximately 500 ml. The containers 100 are interconnected by tubing 50 which can be clamped or sealed as further described below. The first container or primary collection container 100a has tubing and a needle set 81 to allow blood to be drawn from a donor and includes, within the sterile container, anti-coagulant solution 78. Alternatively, the first container 100a can be sterile connected to a unit of whole blood or a blood component prepared by apheresis, in which case anticoagulant is not necessary. The first container 100a can also be attached to other blood sources such as a blood collection apparatus or an infusion apparatus. The collection bag 100a includes a storage container or compartment 102 containing particles 16, as illustrated in FIG. 8A, and described above with respect to FIG. 6. After the blood 12 is collected in the collection bag as illustrated in FIG. 8B, the particles 16 are released from the compartment 102 and allowed to disperse through the blood 12, as illustrated in FIG. 8C.

The collection bag 100A can, in addition, be mixed using a blood container mixer similar to that disclosed in FIGS. 3A and 3B above. In either dispersion or dispersion and mixing embodiment, the particles are then allowed to settle. The collection container 100a and the associated other containers 100b-100d, interconnected by clamped sterile tubing 50, are placed in a centrifuge and rotated at approximately 1300 times the force of gravity for a time period of approximately 5-15 minutes. It is recognized that the settling and centrifugation can be done simultaneously, as described above.

Referring to FIG. 8D, the blood is illustrated separated with the particles 16 having attached target white blood cells 84 at the bottom of the chamber, a layer of red cells 86, a layer of platelets 94 and a layer of plasma 92 at the top. After centrifugation of the blood in the device, the collection container 100a is placed in an extractor device and compressed, similar to above description. The expressor can have two opposing plates, one of which can be notched to contain the buffy coat layer of platelets 94. The plasma portion 92 is transferred to one of the associated bags 100b. The red blood cell portion 86 is moved to another associated bag, such as 100d shown in FIG. 8E which contains the red blood cell preservative 90. Likewise, the layer of platelets 94 is moved to another collection bag 100c. In one embodiment, the tubes 50 extending from the collection bag 100a to the bag 100d extend near the lower portion of the bag 100a and magnets can be used to supplement the density effect of particles 16 to retain the particles 16 within the bag 100a. The white blood cells 84 and particles 16, which are heavier than the blood, are retained in the primary container 100A, thus effectively leukodepleting the blood.

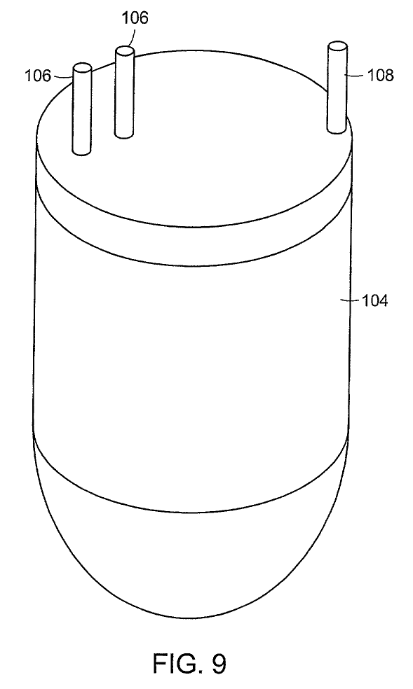

Referring to FIG. 9, a container 104 is shown which is resilient and returns to its nominal shape upon compression or exposure to other distorting force. The container 104 can alternately be rigid. In a preferred embodiment, the base of the container FIG. 9 is either conical or hemispherical to allow collection of the particles 16 in a single location. The container 104 can include one or more inlet tubes 106 to allow the blood 12, and in some embodiments particles 16, into the container 104. In addition, a fluid transfer device or tube 108 is associated with the container 104, preferably is located near an edge of the container 104m such that when the container is rotated, the blood 12, without the white blood cells bound to the particles 16, can be decanted from the container 104 to another sterile container for storage or use.

In one embodiment, the container 104 includes an anti-coagulant solution 78. In one preferred embodiment, the particles 16 are located in a subcompartment within the container 104 such that after the blood 12 is placed into the container 104 through the tube 106, the particles 16 are layered on top of the blood and allowed to disperse through the blood and then settle to the bottom of the container 104. In a preferred embodiment, a container 104 is used in conjunction with a magnet or other device for retaining the particles 16 at the base of the container 104 and the container is rotated to decant the blood, without the white blood cells which are bounded to the particles 16, into a storage container.

### EXAMPLE 1

Depletion of Neutrophils from Whole Blood with Retention of Red Blood Cells and Platelets

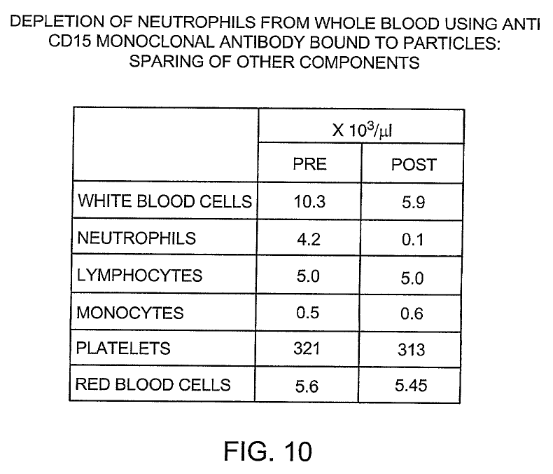

Referring to FIG. 10, anti-coagulated (Na2-EDTA) peripheral blood, 1 mL, was added to 4 mL tube containing particles (15 ul of particles @ 0.5 g/mL) coated with an anti-CD15 monoclonal antibody (KC48 clone, Coulter Corp). The mixture was rotated end over end for 5 minutes, the tube was placed vertically and the particles were allowed to settle by gravity. A magnet was placed at the bottom of the tube and the contents were decanted into a fresh tube.

The pre and post depletion samples were analyzed using a Coulter STKS cell counting instrument that enumerates the various cell populations found in the peripheral blood by size. The results shown in FIG. 10 demonstrate the specific removal of neutrophils and the retention of red blood cells and platelets.

### EXAMPLE 2

Depletion of B-cells in Flexible and Rigid Containers

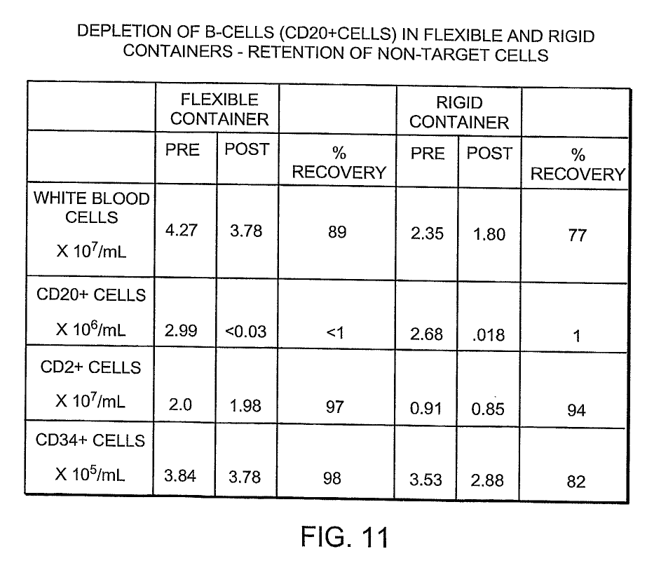

An experiment was performed to compare cell depletion in a flexible bag and a rigid tube container. Referring to FIG. 11, an apheresis leukocyte product was obtained from normal volunteers and the cells were either placed in a flexible 150 mL blood container or a rigid 150 mL conical tube. Particles coated with a monoclonal antibody against B-cells (B1 clone, anti-CD20) had been previously added to the tube at a concentration of 75 uL/mL of blood product to be processed (particles were at concentration of 0.5 g/mL). The containers were rotated for 10 minutes and particles were allowed to settle for 5 minutes. The particles were retained using a magnet and the cellular contents were transferred to a clean container. Samples were taken for cell counts and analysis by flow cytometry measuring T cells (CD2+), B cells (CD20+) and stem cells (CD34+). B-cell depletion was demonstrated to be approximately 2-logs or 99%, the limit of detection of this flow cytometry assay and >90% of non targets were retained.

### EXAMPLE 3

Manufacture of a Sterile Device for Blood Collection and Leukodepletion

A two-container system for blood collection and leukodepletion is manufactured by first making a flexible container from two sheets of polyvinyl chloride. The sheets are sealed together using a Radio Frequency die to heat and seal the seams of the container. Port areas are not bonded to allow for insertion of appropriate elements. Bushings are solvent bonded in the port areas to accommodate blood tubing. The container is filled with an appropriate amount of an anti-coagulant such as acid citrate-dextrose (ACD). Tubing is then solvent bonded onto the bushings. To one tubing lead is attached a needle for a blood draw. The other tubing lead is sealed to create a closed container system. The container with ACD is sterilized by autoclaving at a temperature and duration sufficient to achieve a 10-6 Sterility Assurance Level.

A second container is similarly fabricated from polyvinyl chloride sheeting using a Radio Frequency die, with placement of ports, bushings and tubing leads as above, except that the bottom seam opposite the ports is left open. Particles are added in a fixed amount using a filling device. After filling, the bottom of the container is sealed. The container with particles is sterilized using Ebeam irradiation sufficient to achieve a 10-6 Sterility Assurance Level. Care is taken to maintain a uniform thickness of particles in the container in order to minimize the total Ebeam dose.

The individual containers, one wet and the other dry, are sterile connected using an Ebeam connection. Tubing leads are placed in the field of an Ebeam irradiator while the remainder of the containers are screened from irradiation. A sterile field is created and the tubing leads are cut and reconnected in this field. The resulting device is suitable for use in blood collection and leukodepletion.

While the separation apparatus 10 and a method of use have been described with respect to several blood collection methods, it is recognized that the separation apparatus may be used with other collection system or processing methods for blood. For example, the separation apparatus could be incorporated into other devices such as an autologous blood salvage device which is used in recycling blood from a patient during surgery. The blood salvager suctions free blood from a surgical site and places it in a reservoir. In a typical blood salvager, the blood is filtered using a centrifuge device to remove waste developed at the surgical site, such as bone chips and tissue, and other elements from the healthy red cells. The waste is sent to a storage bag and the red cells are returned to the patient via an intravenous line.

With the separation apparatus 10 as described in the application, the separating apparatus would be placed in line with the reservoir such that the blood that is suction from the surgical field is mixed with particles 16 in a reservoir. The particles 16 with the target cells, such as white cells, are allowed to settle from the remaining blood. The settling of the particles with the white cells can occur before the waste is separated from the blood or, in the alternative, to the red blood cell product after the red blood cell is separated from waste. In both alternates, the white blood cells are separated out from the red blood cells before the blood is returned to the patient.

Likewise, it is recognized that the separating apparatus may be used in a batch, apheresis process where blood is collected from a donor and stored in a reservoir prior to separating a certain component out such as red blood cells and returning the remaining blood components to the donor. In such an embodiment, the separation apparatus 10, as described above, can either be located prior to or after the centrifuge. Depending on the location of the separation apparatus 10, the white blood cells are removed from the whole blood, or the portion that is desired.

While this invention has been particularly shown and described with references to preferred embodiments thereof, it will be understood by those skilled in the art that various changes in form and details may be made therein without departing from the scope of the invention encompassed by the appended claims.

What is claimed is:

1. An apparatus for separating a selected cell population from blood or a blood component, the apparatus comprising:
a container for receiving the blood or blood component;
a plurality of particles having bound thereto a reactant which specifically binds to a selected cell population, the particles having a density sufficient to provide differential gravity settling of the population from the remaining sample, the particle density being at least two times the density of the cells; and
a separating device for separating the selected cell population from the blood or blood component.

2. The apparatus of claim 1 wherein the separating device includes a device for compressing the container.

3. The apparatus of claim 1 wherein the container includes a fluid transfer device for removing fluid from the container.

4. The apparatus of claim 1 wherein the separating device comprises a magnetic device for retaining the particles.

5. The apparatus of claim 1 wherein the separating device comprises a sizing filter for retaining the particles.

6. The apparatus of claim 1 wherein the separating device comprises a flow path allowing the particles to settle from the blood or blood component.

7. The apparatus of claim 1 wherein the separating device comprises a centrifugal device for retaining the particles after differential settling.

8. The apparatus of claim 1 wherein the separating device comprises a centrifugal device for separating the particles from the blood or blood component during differential settling.

9. The apparatus of claim 1 further comprising a device for mixing the particles and the blood.

10. The apparatus of claim 1 further comprising a means for dispersing the particles in blood.

11. The apparatus of claim 1 further comprising a means for retaining the particles prior to introduction into the blood.

12. The apparatus of claim 1 wherein the blood component is whole blood.

13. The apparatus of claim 1 wherein the blood component is red blood cells.

14. The apparatus of claim 1 wherein the blood component is platelets.

15. The apparatus of claim 1 wherein the blood component is plasma.

16. The apparatus of claim 1 wherein the container is generally rigid.

17. The apparatus of claim 16 wherein the separating device includes a magnetic device for retaining the particles and the selected cells bound thereto and a fluid transfer device for decanting the remaining blood or blood component from the container.

18. The apparatus of claim 16 further comprising a device for mixing in the particles and the blood.

19. The apparatus of claim 1 wherein the container is flexible.

20. The apparatus of claim 19 wherein the separating device includes a device for compressing the container.

21. The apparatus of claim 19 wherein the container includes a fluid transfer device for removing a fluid from the container.

22. The apparatus of claim 19 further comprising a centrifuge for spinning the container at a speed to assist in settling of the particles bound to the selected cell population from the remaining blood or blood component.

23. The apparatus of claim 19 further comprising a device for mixing the particles with the blood or blood component.

24. The apparatus claim 23 wherein the device for mixing includes mechanism for rotating the container holding the sample and the particles.

25. The apparatus of claim 1 wherein the particles are formed of nickel.

26. The apparatus claim 1 wherein the particles have a diameter from about 3to 35 microns.

27. The apparatus of claim 1 wherein the particles have a diameter of about 5 microns.

28. The apparatus of claim 1 wherein the particles have a density of about 7-10 g/cm³.

29. The apparatus of claim 1 wherein the particles have a density of about three times the density of the cells of the blood.

30. The apparatus of claim 1 wherein the reactant is an antibody or a fragment thereof.

31. The apparatus of claim 30 wherein the antibody or antibody fragment specifically binds to white blood cells.

32. The apparatus of claim 31 wherein the antibody or antibody fragment is an anti-CD45 antibody.

33. The apparatus of claim 31 wherein the antibody is a pan-leukocyte antibody.

34. The apparatus of claim 1 wherein the reactant is a lectin or a fragment thereof.

35. The apparatus of claim 1 wherein the container comprises a sterile container having a sterilized fluid transfer device providing access to the container.

36. The apparatus of claim 35 wherein the particles are sterilized within the container.

37. The apparatus of claim 1 further comprising a secondary storage container in communication with the container.

38. An apparatus for separating a selected cell population from blood or a blood component, the apparatus comprising:
a flexible primary collection container for receiving the blood or blood component;
a plurality of particles in the primary collection container, the particles having bound thereto a ligand which specifically binds to a selected cell population, the particles having a density sufficient to provide differential gravity settling of the population from the remaining blood components, the particle density being at least two times the density of the cells;
at least one secondary flexible container;
a plurality of fluid transfer devices interconnecting the flexible containers; and
a compression separation device for separating the selected cell population bound to the particles from the blood or blood component, the device moving at least a portion of another portion of the blood through at least one of the fluid transfer devices to at least one of the secondary flexible containers.

39. The apparatus of claim 38 wherein the containers are sterile.

40. The apparatus of claim 38 wherein the separating device further comprises a secondary means for retaining the particles after settling.

41. The apparatus of claim 40 wherein the secondary means comprises a magnetic device.

42. The apparatus of claim 40 wherein the secondary means comprises a sizing filter.

43. The apparatus of claim 40 wherein the secondary means comprises a flow path.

44. The apparatus of claim 40 wherein the secondary means comprises a centrifugal device.

45. The apparatus of claim 38 further comprising a device for mixing the particles and the blood.

46. The apparatus of claim 38 further comprising a centrifuge for spinning at least the primary flexible container at a speed to assist in the settling of the particles from the remaining blood or blood component.

47. The apparatus of claim 38 wherein the ligand is an antibody or fragment thereof.

48. The apparatus of claim 47 wherein the antibody or antibody fragment specifically binds to white blood cells.

49. The apparatus of claim 47 wherein the antibody or antibody fragment is -an anti-CD45 antibody.

50. The apparatus of claim 47 wherein the antibody is a pan-leukocyte antibody.

51. The apparatus of claim 38 wherein the ligand is a lectin or fragment thereof.

52. The apparatus of claim 38 wherein the primary flexible collection container comprises a connection to a blood source.

53. The apparatus of claim 52 wherein the blood source is a donor.

54. The apparatus of claim 52 wherein the blood source is a blood collection apparatus.

55. The apparatus of claim 52 wherein the blood source is an infusion apparatus.

56. The apparatus of claim 38 wherein the primary collection container further comprises a compartment, the particles stored within the compartment prior to binding to a selected cell population.

57. A method of separating leukocytes from whole blood comprising the steps of:
providing a sterile container;
moving blood into the sterile container through a sterile connection;
dispersing a plurality of particles having bound thereto a reactant which specifically binds to leukocytes, the particles having a density sufficient to provide differential gravity settling of the leukocytes from the remaining sample of the whole blood;
settling the leukocytes bound with the particles; and
separating the remaining blood from the leukocyte bound particles to another sterile container.

58. The method of claim 57 wherein the dispersing of the particles is accomplished by rotating the container.

59. The method of claim 57 wherein the dispersing of the particles is accomplished by allowing the particles to gravity settle through the blood.

60. A method of filtering leukocytes from whole blood resulting in high yields of both platelets and red blood cells, the method comprising the steps of:
providing a sterile container;
moving blood into the sterile container through a sterile connection;
dispersing a plurality of particles having bound thereto a reactant which specifically binds to leukocytes, the particles having a density sufficient to provide differential gravity settling of the leukocytes from the remaining sample of the whole blood;
settling the leukocytes bound with the particles; and
decanting the remaining of the blood from the leukocytes bound particles to a second sterile container.
